SEQUENCE LISTING (5 sequences)

SEQ ID NO: 1 - DNA, 8299 bp, artificial sequence
  Plasmid pDAu703
taggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggcctccatggccgcggccgcgctttgctaaaactttggttgatggaaggtatctggcgataaactccgacgacgtctagaagcaacaatcttatgcaaacgctcattggttcttttcgaccgcaacatccatcatgaaactggtattttgtctgtgtcagcagtctagaaccccttgccgggtattttagcatttcatttttctataaaaaggtaccagcatgtatggatcgtatcttccgtaccgtggttattaaatcccagcagaggccgataggcttaagaagtgaacatggcatggttaaggaagaagccattactgagtatatatggctagaataatcgctgggaaagatttatgcttccaagaggcgtaggacggtataccatacagtacggtatttatgaacaattcgataataccactccccaaagcgggagataggacacccgcctcaggcaccaaccaccccctttttcaactgtcagtggtgcacgtttccatcgagcataagcttggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggttgaagttcctattccgagttcctattcttcaaatagtataggaacttcattaattaaaggagagagttgaacctggacgccgcgcaaaaagcaaagacgcgcctcgtgggcggtggatcaatgatcggatttagtggcagatggcatcacaggcggccaatgaccaccgggccaactggccccgacattccagcaatactgcctaattgactccaccatgcatctcggctattattgaactgggtttgatggatggggaccctcttggaattgtcaaagattttgaagcgaagacgatctattggacggtagagatatactcttgatttagtcgttgggaggcccctggggaaagcaatgatggggaatgttgctgctccactgtggacctcggctatggaattacgtgcttggatctaagatgagctcatggctatgcattgaatgacagtgatatcagcagagcaagcagagaaggatggaatgctaattttctagtgctttgtgcaagggtaaatcagggactgtctgtctggtcttctacacgaaggaaagaccatggctttcacggtgtctgtatttccggatatcctcaattccgtcggtcgattacaatcacatgacttggcttccatttcactactattatgcacacccactacatacatgatcatataaccaattgccctcatccccatcctttaactatagcgaaatggattgattgtctaccgccaggtgtcagtcaccctctagatctcgagctcgctagagtcgacctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatcaccgatttctttgggaaatcaataacgctgtcttccgcaggcagcgtctggactttccattcatcagggatggtttttgcgaggcgggcgcgcttatcagcggccagttcttcccaggattgaggcatgtgcatgcaatgtgtgtttatgtggaagtaagatacgacgagtttgattgagaaaagacagggtgattgtcaagttcagtatggaagaaagagtagaagaagatcagacgacagggaagagcgatgacataaaaggtggaagacggaagaaaaacgaaccaaatcaatcccactctatggcgggggttggactgcctgaggccggcactggtggggcttatcgataagttctcgtcaccggatgcaatgcgctgtcaactgctgacttggccctgaacatcctgtcctctacagatccatactatacaatgatcccagttatagtgcggtaaggtgcatatcatatctcattctcatgactcattcgacttttttttagagaaagtacatacgtggaacatacactaaacgcaacaggtcgcgacaacactggtatacaaaacggtccccggtgaatgacgttattagtgtctatcccccactcacacccgaaaagaataatagaaactaacagaaaaagcggcccgaggataagaggaacattcaaacagaaggggaatcataaaaaccgaaaaatgcaaggaaaagagaactcaaatcaataattttcataatactgtcgagagtaatacggaccagcgtctctcagggacatgcgtcggcgcaaggcatcatccaatctctcatctaacacatccagcattcgtgttcgatagtctaactgcttctctcggcgctcaagtcttgcttcccgatcatcgagttaattaagaagttcctatactttctagagaataggaactcggaataggaacttcaaggtaccgagctctatcctcaataccctattttccacgattccattgtcatatccaattccgttttcttttcttgttttcccctcatccaatcccgtccatcatttactcctttttcttgtgaatgcaagtggcactaagaaatccaacccccagacaaattttcctactcaggaacacaaaaacctcgtttctgctcccttctcgtacttcattcctatcgtctcggaatttcctcaacaaccctttccgactttgcgacagcgtcgcgattccagacttatgtgttctcgttcctactgtcgttaccagtctatttattccgaaacctctgatcgctgaatttcacacacaacacccccccgttgatgctggtggagaatccgtagcgtcaagagttgaattcactccatgttgtaacgaagtccacgaattgagacgattgatgattacaaccccgcgatcgcctatcgacgattcgacgagatgccattctcatcctcctcatcctcctccacccccgaggtgtctaccaccccgctcgcagattacttctggatcgcaggtgtcgatggcgcggaaatcttagagactttccaaagactcggcgacgaatacagggcaaacagtgccaccgctcctggccccgctcttgcggacacgatcgaggaagatgcggacgcggaggaggcacacgacccccgtctggactccctctctcgacccaattccatggctgggggccgcaattccttccagcggttctcaatgcgctcaggagactccagtgagtccagtgggaatggtaccagcagcaaccggagcagtctgaccatcaagggtaatcagtcgcccagagggtcgtcgtttctagaagatttcgactttgacaaggccctgttcaagtttgcaaacgagcgggagtcgttcctgtcggatctgagtctcagtgccggagcaatcactcccacctcccgtcctaggtccaggttacgtacacagaagattgtctccgaggaaagtccctcccagccatccagcttgcttcgatcaggcattggtagtgtgcggcgtcatatggcattcagagacatgaatagtatgaaacggcagccgtcagttgctcgtcgcggccgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaa

SEQ ID NO: 2 - DNA, 8614 bp, artificial sequence
  Plasmid pDAU724
gaattcgagctcggtaccttgaagttcctattccgagttcctattctctagaaagtataggaacttcagtacccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatgaggagctcccttgtgctgttctttgtctctgcgtggacggccttggccagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttagtggccggcgcggctgggtcgactctagcgagctcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaatgtacattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcattgaagttcctatactatttgaagaataggaactcggaataggaacttcaacgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagtaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 3 - DNA, 8712 bp, Artificial sequence
  Plasmid pBac7000
  misc_recomb (19)..(67): FRT-F
  misc_binding (91)..(104): amyR
  misc_binding (112)..(125): amyR
  misc_binding (133)..(146): amyR
  misc_binding (153)..(166): amyR
  misc_binding (174)..(187): amyR
  misc_binding (195)..(208): amyR
  promoter (219)..(832): In vivo promoter
  misc_binding (347)..(360): amyR
  misc_binding (568)..(581): amyR
  misc_binding (619)..(632): amyR
  misc_binding (640)..(653): amyR
  misc_RNA (767)..(860): 5′ untranslated region
  CDS (845)..(913): Signal peptide
  Intron (914)..(1029): Intron with T7 promoter
  promoter (949)..(1021): T7 promoter with Shine-Dalgarno sequence
  RBS (1016)..(1021): Shine-Dalgarno sequence
  CDS (1030)..(1845): lipase mature peptide encoding region
  terminator (1861)..(2551)
  misc_recomb (3569)..(3618): FRT-F3 on complementary strand
gaattcgagctcggtaccttgaagttcctattccgagttcctattctctagaaagtataggaacttcagtacccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatgatgaggagctcccttgtgctgttctttgtctctgcgtggacggccttggccagtcctattcgtcgagaggtatgtacaccacccccttgcgtctgatctgtgactaatacgactcactatagggagaccacaacggtttccctctagaaataattttgtttaactttaagaaggagaagctgactatggaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttagtagtctcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaatgtacattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcattgaagttcctatactatttgaagaataggaactcggaataggaacttcaacgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagtaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 4 - protein, 23 aa, Artificial sequence
  Synthetic Construct
MRSSLVLFFVSAWTALASPIRRE

SEQ ID NO: 5 - protein, 270 aa, Artificial sequence
  Synthetic Construct
MEVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

(19) United States
(12) Patent Application Publication
(10) Pub. No.: US 2019/0078097 A1
(43) Pub. Date: Mar. 14, 2019

(54) Polynucleotide Constructs For In Vitro and In Vivo Expression

(71) Applicant: Novozymes A/S, Bagsvard, (DK)

(72) Inventors: Charles Emrich, San Francisco, CA (US); Jesper Vind, Vaerlose (DK)

(73) Assignee: Novozymes A/S, Bagsvaerd (DK)

(21) Appl. No.: 15/772,489

(22) PCT Filed: Oct. 28, 2016
(86) PCT No.: PCT/US2016/059427

§ 371 Date: Apr. 30, 2018

Related U.S. Application Data
(60) Provisional application No. 62/248,553, filed on Oct. 30, 2015.

(51) Int. Cl. C12N 15/52; C12N 15/75; C12N 15/80; C12N 9/90; C12N 9/00; C12N 9/88; C12N 9/02; C12N 9/10; C12N 9/40; C12N 9/34; C12N 9/38; C12N 9/48; C12N 9/42; C12N 9/26

(52) U.S. Cl. CPC C12N 15/52; C12N 15/75; C12N 15/80; C12N 9/90; C12N 9/93; C12N 9/88; C12N 9/2431; C12N 9/10; C12N 9/2465; C12N 9/2428; C12N 9/2468; C12N 9/48; C12N 9/2437; C12N 9/0004

(57) ABSTRACT

The present invention relates to polynucleotide constructs for in vitro and in vivo transcription/translation of genes of interest or variants of a gene of interest as well as microorganism host cells comprising such constructs and methods for producing a polypeptide of interest in such microorganism host cells.

### REFERENCE TO A SEQUENCE LISTING

This application contains a Sequence Listing in computer readable form, which is incorporated herein by reference.

### FIELD OF THE INVENTION

The present invention relates to polynucleotide constructs for in vitro and in vivo transcription/translation of genes of interest or variants of a gene of interest as well as microorganism host cells comprising such constructs and methods for producing a polypeptide of interest in such microorganism host cells. The constructs of the invention allow the in vitro transcription/translation (IVTT) production of the encoded desired protein(s) as well as direct transformation of the constructs into fungal host cells for in vivo production of the secreted protein(s).

The promoter driving the in vitro transcription is situated in an intron just upstream of the gene encoding the mature protein of interest. Upstream of the intron is a polynucleotide encoding a secretion signal peptide which is expressed from a fungal promoter in vivo.

### BACKGROUND

Successful in vivo production of a secreted polypeptide of interest in a microorganism having a secretory machinery often depends on the expression of a signal peptide in translational fusion with the polypeptide.

However, in some situations it is preferred to express a polypeptide of interest in vitro. This is typically done with a cell-free cell extract that contains all the prerequisites for DNA transcription and translation. Obviously, a polypeptide of interest that is produced in an in vitro transcription translation (IVTT) system does not need a signal peptide, as it will be produced directly in the cell-free medium. So, if a pro- or a pre-pro form of the polypeptide is normally produced in vivo, only the mature polypeptide-encoding polynucleotide would need to be expressed in the IVTT system to produce the mature polypeptide.

If a signal peptide were included in the in vitro expression construct, it would likely not be cleaved off like it would be during in vivo secretion, and the presence of the signal peptide would change the characteristics of the polypeptide as compared to the mature polypeptide. The presence of a signal peptide could, thus, also have undesirable effects on a screening of the in vitro expressed polypeptide.

This means, of course, that separate polynucleotide constructs appear to be needed to provide either in vivo or in vitro transcription and translation to produce the polypeptide of interest.

The present invention is based on the fact that many microorganisms, including bacteria and fungal host cells, are capable of processing so-called introns. An intron is any nucleotide sequence within a gene that is removed by RNA-splicing during maturation of the final messenger-RNA product. When proteins are encoded by intron-containing genes, RNA-splicing takes place as part of the RNA processing pathway that follows transcription and precedes translation.

### SUMMARY OF THE INVENTION

The inventors successfully introduced an artificial intron into a polynucleotide construct comprising a fungal promoter operably linked to a polynucleotide encoding a signal peptide in translational fusion with a mature lipase.

The artificial intron was introduced in the polynucleotide construct between the signal-peptide coding sequence and the mature lipase-encoding sequence, so that it would be excised from the transcribed RNA during mRNA maturation in a fungal host cell to produce a mRNA encoding the signal peptide in correct translational fusion with the mature lipase.

When the resulting intron-containing polynucleotide construct was transformed into a fungal host, the mature lipase was produced and secreted. This result demonstrated that the fungal host cell had transcribed the full RNA sequence, then removed the intron from the RNA during mRNA maturation and translated the mRNA into the encoded signal peptide in correct fusion with the lipase which was, in turn, secreted by the cell.

Before its integration into the construct, the artificial intron was engineered to contain a promoter known to be active in an in vitro transcription translation (IVTT) system, so that this promoter would be operably linked with the mature lipase-encoding sequence once the intron had been introduced into the construct.

When the resulting intron-containing polynucleotide construct was mixed with an IVTT system, the active mature lipase was produced directly in vitro.

In this way, the inventors have surprisingly provided proof of concept that a single polynucleotide construct can be ingeniously put together to allow the production of an active polypeptide, irrespective of whether it is produced directly in an IVTT system or secreted by a microorganism host cell in vivo.

Accordingly, in a first aspect the invention relates to isolated polynucleotide constructs comprising the following elements in 5′ to 3′ order:

(a) a first polynucleotide having promoter activity in a microorganism host cell capable of processing an intron, wherein the first polynucleotide is operably linked with a signal peptide-encoding polynucleotide;

(b) an intron comprising a second polynucleotide having promoter activity in an in vitro transcription/translation (IVTT) system; and

(c) a third polynucleotide encoding a polypeptide of interest operably linked with the first and second polynucleotides of (a) and (b);

whereby the first polynucleotide ensures expression of the signal peptide in translational fusion with the polypeptide of interest in the microorganism host cell; and

whereby the second polynucleotide ensures expression of the polypeptide of interest without a signal peptide in the IVTT system.

In a second aspect, the invention relates to microorganism host cells comprising a polynucleotide construct as defined in the first aspect.

In a final aspect, the invention relates to methods for producing a polypeptide of interest, said method comprising the steps of:

a) cultivating a microorganism host cell as defined in the previous aspect; and, optionally

b) recovering the polypeptide of interest.

### BRIEF DESCRIPTION OF THE FIGURES

FIG. 1 shows a schematic drawing of vector pDAu703.

FIG. 2 shows schematic drawings of:
Top part: the amy2-region of the chromosome in A. oryzae host strain DAu716, where two FRT-sites have been inserted;
Middle part: the linearized vector pDAu724; and
Bottom part: the amy2-region of the chromosome in A. oryzae host strain DAu716 after FLP-mediated integration of pDAu724 by double-homologous recombination between the respective FRT-sites.

FIG. 3 shows a schematic overview of a polynucleotide construct of the invention depicted with the T7 bacteriophage promoter as the promoter for the in-vitro transcription translation system.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment×Total Number of Gaps in Alignment)

### DETAILED DESCRIPTION OF THE INVENTION

### Polynucleotide Constructs

In a first aspect, the invention relates to isolated polynucleotide constructs comprising the following elements in 5′ to 3′ order:

(a) a first polynucleotide having promoter activity in a microorganism host cell capable of processing an intron, wherein the first polynucleotide is operably linked with a signal peptide-encoding polynucleotide;

(b) an intron comprising a second polynucleotide having promoter activity in an in vitro transcription/translation (IVTT) system; and

(c) a third polynucleotide encoding a polypeptide of interest operably linked with the first and second polynucleotides of (a) and (b);

whereby the first polynucleotide ensures expression of the signal peptide in translational fusion with the polypeptide of interest in the microorganism host cell; and

whereby the second polynucleotide ensures expression of the polypeptide of interest without a signal peptide in the IVTT system.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used. Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

In a preferred embodiment of the invention, the first polynucleotide has promoter activity in a bacterial host cell; preferably in a prokaryotic host cell; more preferably in a Gram-positive or Gram-negative bacterium; more preferably in a Bacillus host cell; even more preferably in a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cell; most preferably in a Bacillus subtilis or Bacillus licheniformis cell.

In another preferred embodiment, the first polynucleotide has promoter activity in a fungal host cell. Preferably the fungal host cell is be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may preferably be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The preferred filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

Most preferably, the filamentous fungal host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In a preferred embodiment, the fungal host cell is a filamentous fungal host cell, more preferably an Aspergillus or Trichoderma host cell; most preferably an Aspergillus oryzae, Aspergillus niger or a Trichoderma reesei cell.

In a preferred embodiment of the invention, the first polynucleotide has promoter activity in a fungal host cell and comprises or consists of a promoter derived from an Aspergillus or a Trichoderma cell; more prefably the first polynucleotide comprises or consists of a promoter derived from an Aspergillus oryzae, Aspergillus niger or a Trichoderma reesei cell; even more preferably the first polynucleotide comprises or consists of a fungal triose-phosphate isomerase promoter of an Aspergillus oryzae, Aspergillus niger or a Trichoderma reesei cell; most preferably the first polynucleotide comprises or consists of the promoter shown in positions 219-838 of SEQ ID NO:3.

In another preferred embodiment, the signal peptide is derived from a bacterial signal peptide; preferably the signal peptide is derived from a prokaryotic cell; more preferably the signal peptide is derived from a Bacillus cell.

In an alternative preferred embodiment, the signal peptide is derived from a fungal cell; preferably the signal peptide is derived from a filamentous fungal cell; even more preferably the signal peptide is derived from an Aspergillus or a Trichoderma cell; most preferably the signal peptide is derived from an Aspergillus oryzae, Aspergillus niger or a Trichoderma reesei cell.

Preferably, the second polynucleotide comprises or consists of a bacterial promoter, preferably the second polynucleotide comprises or consists of a promoter from a bacteriophage, most preferably the second polynucleotide comprises or consists of the T7 promoter shown in positions 949-1021 of SEQ ID NO:3.

As exemplified below with a lipase enzyme, it is preferred that the third polynucleotide encodes a enzyme; preferably a hydrolase, isomerase, ligase, lyase, oxidoreductase, or transferase; even more preferably an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase; preferably the third polynucleotide encodes the mature form of the enzyme.

### Host Cells

In a second aspect, the invention relates to microorganism host cells comprising a polynucleotide construct as defined in the first aspect.

A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In a preferred embodiment the microorganism host cell is a bacterial host cell; preferably a prokaryotic host cell; more preferably a Bacillus host cell; most preferably a Bacillus subtilis or Bacillus licheniformis cell.

In an alternative embodiment the microorganism host cell is a fungal host cell; preferably a filamentous fungal host cell, more preferably an Aspergillus or a Trichoderma host cell; most preferably an Aspergillus oryzae, Aspergillus niger or a Trichoderma reesei cell.

### Methods of Production

A final aspect of the invention relates to methods for producing a polypeptide of interest, said method comprising the steps of:

a) cultivating a microorganism host cell as defined in the previous aspect; and, optionally

b) recovering the polypeptide of interest.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide. The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### EXAMPLES

### Example 1

### Construction of a Split-Marker Aspergillus Oryzae Host/Vector System

One way to ensure the proper orientation when integrating an expression vector by flippase-mediated site-specific recombination into a suitable fungal host cell is to employ a split selection marker, where one non-functional part of the marker resides in the host chromosome and another non-functional part of the marker is on the incoming vector. Only the correctly oriented integration then results in a functional second selection marker. That split-marker principle is illustrated in this example; here the second selection marker is oriented in one direction but it could just as well have been oriented the other way.

### Media and Solutions Necessary for Aspergillus Protoplast Transformation and Selection of Recombinant Cells:

### Trace Metal

| | |
|---|---|
| Na₂B₄O₇•10aq | 40 mg/l |
| CuSO₄•5aq | 400 mg/l |
| FeSO₄•7aq | 1200 mg/l |
| MnSO₄•aq | 700 mg/l |
| Na₂MoO₂•2aq | 800 mg/l |
| ZnSO₄•7aq | 10.000 mg/l |

### Salt Solution

| | |
|---|---|
| KCl | 26 g/l |
| MgSO₄•7aq | 26 g/l |
| KH₂PO₄ | 76 g/l |
| Trace metal | 50 ml/l |

### COVE Medium

20 ml salt solution

20 g agar

218 g sorbitol

H₂O ad 1 l

Autoclave and then add:

50 ml 20% glucose

10 ml 1M urea.

### Cove-N-gly Slant

| | | |
|---|---|---|
| Salt solution | 50 | ml |
| Sorbitol | 218 | g |
| kaliumnitrat | 2.02 | g |
| Glycerol | 10 | ml |
| Agar | 35 | g |
| MilliQ H₂O to | 1000 | ml |

### Sucrose Medium

20 ml salt solution.

342 g sucrose.

H₂O ad 1 l

Autoclave and then add:

10 mM NaNO3
ST 0.6 M sorbitol

100 mM Tris/HCl pH 7.0
STC 1.2 M sorbitol

10 mM CaCl₂

10 mM Tris/HCl pH 7.5.
PEG 60% (W/V) PEG 4000 (BDH) (6 g PEG+-5 ml sterile water, put at 60-65° C.)

10 mM CaCl₂ (50 μl of a 2 M CaCl₂)

10 mM Tris/HCl pH 7.5. (100 μl of a 1M Tris)

### Sucrose Agar Plate

10 g Agar

10 ml Salt solution

1M sucrose to 500 ml

Autoclave to sterilized

### Acetamide Plates

10 ml salt solution

10 g agar

1M sucrose ad 500 ml¹.

autoclave.

Cool to approx. 65° C. and add 10 mM acetamide and 15 mM CsCl.

Triton X-100 50 μl for 500 ml (only in the restriking plates)

Introduction of the FRT Sites at the amy2 Locus in Aspergillus Oryzae DAu716

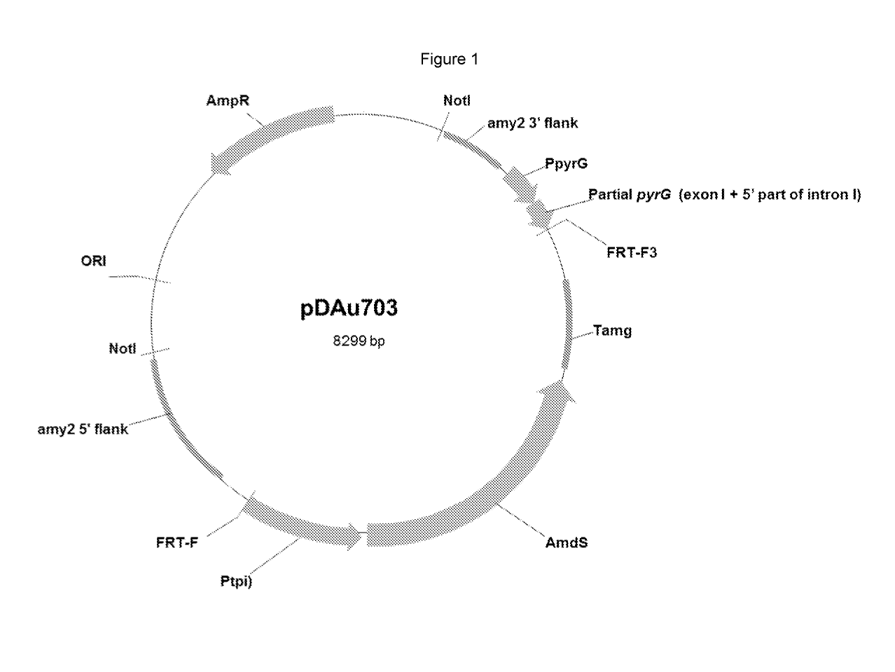

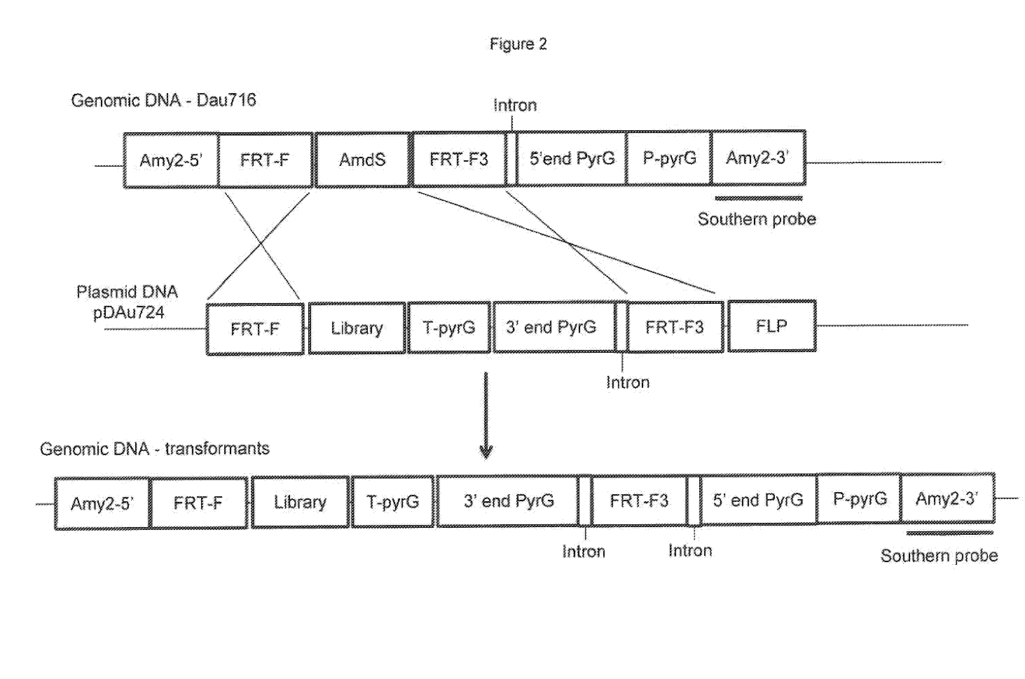

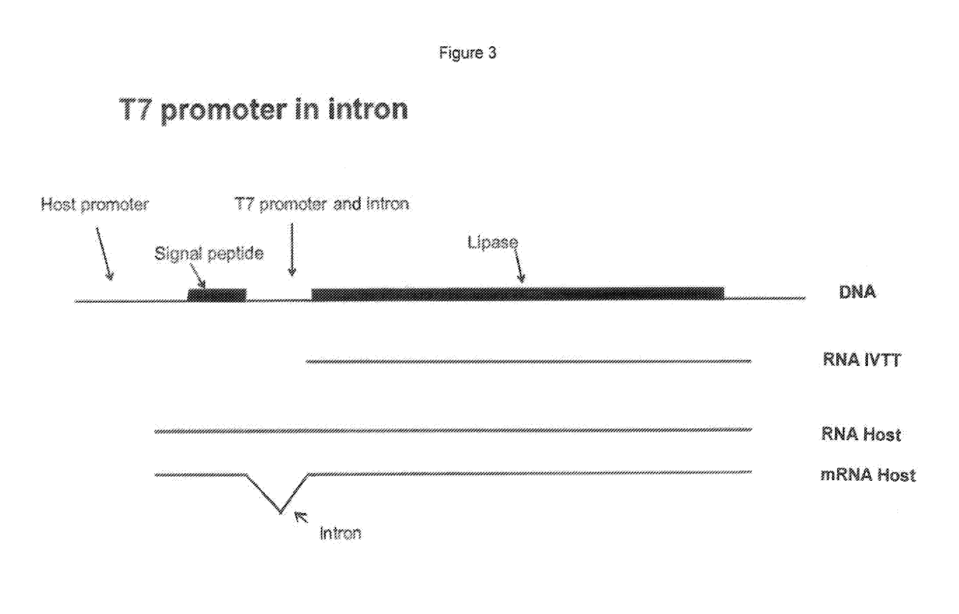

The plasmid pJAI1258 (described in WO12160097A1) was modified resulting in a plasmid denoted pDAu703. Plasmid pDAu703 contains the following elements in order (FIG. 1; SEQ ID NO:1):
amy2-3′ flank (490 bp); positions 449-938 of SEQ ID NO:1;
pyrG promoter operably linked with a partial pyrG gene containing the 5′-end of the pyrG CDS (the first exon and 5′ end of its first intron);
a FRT-F3 site (50 bp); positions 1452-1501 of SEQ ID NO:1;
an A. niger AMG terminator (Tamg) operably linked with the AmdS-encoding gene, positions 1511-2200 of SEQ ID NO:1;
A. nidulans acetamidase gene (AmdS), positions 2232-4131 of SEQ ID NO:1;
the strong triose-phosphate isomerase promoter (Ptpi) operably linked with the Amds-encoding gene; this allows growth on acetamide and CICs even though only one copy of the AmdS selection cassette is present in the genome as expected if the plasmid pDAU703 is integrated in one copy at the amy2 locus at FRT sites. Positions 4140-4894 of SEQ ID NO:1;
a FRT-F site (49 bp); positions 4903-4951 of SEQ ID NO:1;
amy2-5′ flank (1114 bp); positions 4964-6077 of SEQ ID NO:1;
The rest of the plasmid is composed of a part of DNA necessary for the maintenance of the plasmid as a replicative plasmid in the bacterial host cell E. coli (E. coli origin of replication and ampicillin resistance cassette).

Plasmid DNA pDAu703 was digested with NotI restriction enzyme to separate the DNA containing the integration cassette from the now irrelevant E. coli part of the plasmid.

The linearized plasmid pDAu703 was transformed into protoplasts of A. oryzae strain Ja11338 (disclosed in WO12160097A1) using a standard procedure described, for example, in WO98/01470 but with supplementing the media with 10 mM uridine since the strain is PyrG minus and therefore cannot grow in absence of uridine. Transformants were selected on AmdS selection plates

The resulting recombinant host strains have had the two FRT sites as well as the 5′ end of the split PyrG marker (first exon and part of the native intron) operably linked with its own promoter integrated by homologous recombination at the amy2 locus, as shown in the top panel of FIG. 2. The correct integration at the amy2 locus was checked by Southern blot analysis using a probe that annealed to the amy2 3′ end (FIG. 2). Integration of the FRT cassette generated hybridization signals at 5114 bp and 2637 bp in EcoRI and XhoI digests, respectively (not shown). This pattern is different from the Ja11338 host, where the amy2 locus is not disrupted. A correct strain was selected and denoted A. oryzae DAu716 (FIG. 2, top).

Transformation of DAu716 with the pDAU724 Vector Carrying a Lipase-Encoding Gene

This example demonstrates how the FRT/FLP recombination and split PyrG marker can be used to effectively make single copy insertions of an expression cassette with a high frequency in A. oryzae. We used the lipase gene from Thermomyces lanuginosa (e.g. disclosed in WO2008008950) as a reporter to measure the level of lipase produced in a transformed host.

Like in the previous example, an expression vector was constructed so that part of it can be integrated into the chromosome of the host cells at the FRT-sites using flippase as site specific recombination mediator. The part of the plasmid that is to be integrated in the genome carries a lipase gene operably linked with the NA2/TPi promoter and the terminator of the A. niger AMG gene. In order to be able to select the recombinant cells that have successfully integrated the expression cassette via the FRT sites, the remainder of the pyrG selection marker is also included in between the FRT sites. The promoter and the first exon resides in the DAu716 host and the remainder of the pyrG marker resides on the incoming plasmid. Upon site specific recombination, the PyrG marker will be reconstructed as an intact gene (with a FRT sequence inside its first intron which will, of course, be spliced out from the mRNA) and the recombinant cells will be able to express PyrG and grow on plate with NaNO₃ as sole nitrogen source.

Plasmid pDAU724 (FIG. 2, middle; SEQ ID NO:2) consists of:

PART-I which is to be integrated in the genomic DNA of the Aspergillus host cells and it consists of the two FRT sites with the expression cassette and one part of the split pyrG marker;

PART-II which will not be integrated in the genome of the host cell and which contains the FLPase expression cassette as well as E. coli selection marker and origin of replication.

The strain DAu716 was grown on a slant of Cove-N-gly medium until spores could be seen.

10-20 ml of Sucrose medium or YPD medium was added to the slant, and the spores were suspended by vortexing the slant. The spore suspension was transferred to a polycarbonate shakeflask (500 ml) containing 100 ml sucrose medium with 10 mM NaNO₃ (or other nitrogen source). The flask was incubated at 30° C. for 24 hr (200 rpm).

The mycelium was collected by filtration through miracloth and washed using 200 ml 0.6 M MgSO₄. The remaining liquid was squeezed out of the mycelium e.g. using a plastic pipette.

1-2 g of the mycelium was transferred to a small (100 ml) polycarbonate flask containing:

75-150 mg Glucanex

10 ml 1.2 M MgSO₄

100 ul 1 M NaH₂PO₄ pH 5.8

and the mycelium was suspended, 1 ml of 12 mg/ml BSA (sterile filtered) was added

The suspension was incubated at 37° C. for ½-2 hr, and the protoplasting was monitored frequently by microscopy.

The protoplast suspension was filtered through miracloth into a 25 ml centrifuge tube and the suspension was overlaid with 5 ml ST (being careful not to mix up the lower layer). The resulting protoplasts were banded by centrifugation (2500 rpm/1350 g, 15 min, slow acceleration). The interface band of protoplasts was recovered and transferred to a fresh tube.

The protoplasts were diluted with 2 volumes of STC followed by centrifugation (2500 rpm/1350 g, 5 min). The protoplasts were then washed twice with 5 ml STC (using resuspension and centrifugation), and then resuspended in STC to a concentration of approx 5×10⁷ protoplasts/ml.

For each transformation, the transforming DNA was added at the bottom of e.g. a 14 ml tube, and 100 μl of protoplasts were added. 300 μl of PEG was added, and the tube was gently mixed by hand. After 20 minutes of incubation (RT), 6 ml top agar at temperature of 50° c was added and immediately the suspension was poured on to a selective sucrose agar plate with 10 mM Na NO_{3.}

The plates were incubated at 37° C. until transformants were clearly visible and started to sporulate. 20 transformants were restriked onto a new selection plate with triton to isolate colonies that could be further analyzed by fermentation, Southern blot analysis or enzyme activity assay.

It was verified that the residing AmdS marker in the chromosome had been replaced by the incoming lipase gene in the transformants by streaking the transformants on plates containing CsCl (an inhibitor of the endogenous acetamidase) and acetamide as sole nitrogen source. Correct transformants should not be able to grow on these plates. We tested 20 recombinant cells obtained after transformation of pDAu724 into DAu716 and only a slight growth phenotype was observed compared to the parent host strain DAu716, where the AmdS selection marker is still present.

It was confirmed that all 20 transformants contained one inserted copy of the lipase expression cassette correctly inserted at the FRT sites.

The 20 transformants were inoculated in 3 ml YPD in a Uniplate® 10 ml 24 deep-wells plate (Whatman) sealed with Airpore tape (Quiagen) and incubated at 30 degree Celcius for 4 days with 200 rpm agitation. The supernatants were collected for further analysis (lipase assay and SDS-page) and the mycelia were also collected for genomic extraction and Southern analysis.

The 20 transformants showed comparable lipase activities in a lipase assay as well as comparable lipase protein levels on an SDS-PAGE gel (not shown). In addition, a Southern blot confirmed that all 20 transformants had only the expected single lipase gene copy correctly integrated in the chromosome (not shown).

### Example 2

### Construction of Plasmid pBac7000

The plasmid pDau724 containing a Thermomyces lanuginosus lipase as reporter gene was used as vector. The plasmid has been constructed such that is contains a flippase gene which ensures homologous recombination into the host genome using the Frt-sites flanking the gene of interest, as shown in the above example.

An intron containing the T7 promoter in the fungal secreted lipase-encoding gene was ordered as a synthetic polynucleotide construct and cloned into pDau724 using restriction enzyme BamHI and XhoI using standard molecular biology techniques, thus creating plasmid pBac7000 (SEQ ID NO:3).

### Example 3

### In Vitro Transcription/Translation of Gene Encoding a Lipase

pBac7000 and pDau724 were used in IVTT reactions using a standard kit from Biolabs (PURExpres In vitro Protein synthesis kit E6800S). No lipase expression was seen from pDau724 but nice expression of lipase was seen using pBac7000 as template. The expression was tested in an activity assay using PNP-valerate as substrate (details disclosed in WO200024883).

### Example 4

### Transformation of and Expression in Fungal Host with Gene Encoding a Lipase

pBac7000 and pDau724 were each transformed separately into strain Dau716 cells. The transformants were inoculated into 96 well microtiter plate (MTP) well each containing 200 microliter YPM media. The MTP was grown for 3 days at 34° C. without shaking in a small box with wet paper to ensure high humidity. 10 microliter of growth media from each well was assayed for lipase activity using the PNP-valerate assay mentioned in the previous example. Both strains gave comparable level of lipase activity (data not shown).

1. An isolated polynucleotide construct comprising the following elements in 5′ to 3′ order:
(a) a first polynucleotide having promoter activity in a microorganism host cell capable of processing an intron, wherein the first polynucleotide is operably linked with a signal peptide-encoding polynucleotide;
(b) an intron comprising a second polynucleotide having promoter activity in an in vitro transcription/translation (IVTT) system; and
(c) a third polynucleotide encoding a polypeptide of interest operably linked with the first and second polynucleotides of (a) and (b);
whereby the first polynucleotide ensures expression of the signal peptide in translational fusion with the polypeptide of interest in the microorganism host cell; and
whereby the second polynucleotide ensures expression of the polypeptide of interest without a signal peptide in the IVTT system.

2. The polynucleotide construct of claim 1, wherein the first polynucleotide has promoter activity in a bacterial host cell; preferably in a prokaryotic host cell; more preferably in a Bacillus host cell; most preferably in a Bacillus subtilis or Bacillus licheniformis cell.

3. The polynucleotide construct of claim 1, wherein the first polynucleotide has promoter activity in a fungal host cell; preferably in a filamentous fungal host cell, more preferably in an Aspergillus or Trichoderma host cell; most preferably in an Aspergillus oryzae, Aspergillus niger or a Trichoderma reesei cell.

4. The polynucleotide construct of claim 1, wherein the first polynucleotide has promoter activity in a fungal host cell and comprises or consists of a promoter derived from an Aspergillus or a Trichoderma cell; more preferably the first polynucleotide comprises or consists of a promoter derived from an Aspergillus oryzae, Aspergillus niger or a Trichoderma reesei cell; even more preferably the first polynucleotide comprises or consists of a fungal triose-phosphate isomerase promoter of an Aspergillus oryzae, Aspergillus niger or a Trichoderma reesei cell; most preferably the first polynucleotide comprises or consists of the promoter shown in positions 219-838 of SEQ ID NO:3.

5. The polynucleotide construct of claim 1, wherein the signal peptide is derived from a bacterial signal peptide; preferably the signal peptide is derived from a prokaryotic cell; more preferably the signal peptide is derived from a Bacillus cell.

6. The polynucleotide construct of claim 1, wherein the signal peptide is derived from a fungal cell; preferably the signal peptide is derived from a filamentous fungal cell; even more preferably the signal peptide is derived from an Aspergillus or a Trichoderma cell; most preferably the signal peptide is derived from an Aspergillus oryzae, Aspergillus niger or a Trichoderma reesei cell.

7. The polynucleotide construct of claim 1, wherein the second polynucleotide comprises or consists of a bacterial promoter, preferably the second polynucleotide comprises or consists of a promoter from a bacteriophage, most preferably the second polynucleotide comprises or consists of the T7 promoter shown in positions 949-1021 of SEQ ID NO:3.

8. The polynucleotide construct of claim 1, wherein the third polynucleotide encodes a enzyme; preferably a hydrolase, isomerase, ligase, lyase, oxidoreductase, or transferase; even more preferably an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

9. The polynucleotide construct of claim 8, wherein the third polynucleotide encodes the mature form of an enzyme.

10. A microorganism host cell comprising a polynucleotide construct as defined in claim 1.

11. The microorganism host cell of claim 10 which is a bacterial host cell; preferably a prokaryotic host cell; more preferably a Bacillus host cell; most preferably a Bacillus subtilis or Bacillus licheniformis cell.

12. The microorganism host cell of claim 10 which is a fungal host cell; preferably a filamentous fungal host cell, more preferably an Aspergillus or a Trichoderma host cell; most preferably an Aspergillus oryzae, Aspergillus niger or a Trichoderma reesei cell.

13. A method for producing a polypeptide of interest, said method comprising the steps of:
a) cultivating a microorganism host cell as defined in claim 10; and, optionally
b) recovering the polypeptide of interest.
